(19) United States
(12) Patent Application Publication
(10) Pub. No.: US 2015/0290225 A1
(43) Pub. Date: Oct. 15, 2015

(54) COMPOSITIONS AND METHODS OF USING SAME

(71) Applicant: Dignity Sciences Limited, Dublin (IE)

(72) Inventors: John Climax, Dublin (IE); Mehar Manku, Birmingham (GB); David Coughlan, Dublin (IE)

(21) Appl. No.: 14/682,966

(22) Filed: Apr. 9, 2015

Related U.S. Application Data
(60) Provisional application No. 61/977,231, filed on Apr. 9, 2014.

(51) Int. Cl. A61K 31/661; A61K 31/355; A61K 31/375; A61K 31/365

(52) U.S. Cl. CPC A61K 31/661; A61K 31/365; A61K 31/355; A61K 31/375

(57) ABSTRACT

The present disclosure provides compositions and methods for the treatment of disease and/or disorders of this skin.

### PRIORITY CLAIM

This application claims priority to U.S. Provisional Patent Application Ser. No. 61/977,231 entitled “COMPOSITIONS AND METHODS OF USING SAME,” which was filed on Apr. 9, 2014, the entire contents of which are incorporated herein by reference and relied upon.

### FIELD

The disclosure generally relates to compositions for the treatment of diseases and/or disorders such as acne or related skin conditions.

### BACKGROUND

There are over a dozen subtypes of acne. Most types of acne are caused by environmental exposure, chemical exposure, physical trauma or abrasion of the skin, or sensitivity to hormone levels. One type, acne vulgaris, and related forms of acne including, for example, acne necrotica, has been linked to bacterial infection by Propionibacterium acnes. Presently, there exist numerous regimens for the treatment of acne including topical application of creams comprising benzoyl peroxide. However, such creams are not always effective at reducing the growth of bacteria and ameliorating the clinical manifestation of the condition. Additionally, current regimens are limited in dose strength due to side effects. Accordingly, there exists a need for compositions that are more effective in the treatment of acne.

### SUMMARY

The present disclosure provides compositions used singly or in combination with anti-bacterial agents for the treatment of disease and/or disorders such as acne or atopic dermatitis or for cosmetic use.

The present disclosure also provides methods for treating or preventing acne in a subject in need thereof comprising administering to the subject a composition comprising one or more of ascorbyl palmitate, tocopherol, ascorbic acid and lecithin. In some embodiments, ascorbyl palmitate, tocopherol, ascorbic acid and lecithin are each, if present, present in an amount of about 0.01% to about 5%, about 0.05% to about 2.5% or about 0.075% to about 1%, by weight of the composition.

In some embodiments, the compositions comprises one or more excipients. Such excipients can be pharmaceutically acceptable or cosmetically acceptable excipients.

In some embodiments, the compositions further includes a therapeutically effective amount of benzoyl peroxide. In some embodiments, the composition comprises about 1.25 wt. % to about 10 wt. % of benzoyl peroxide.

In some embodiments, the composition further includes a therapeutically effective amount of benzoyl peroxide. In some embodiments, the composition comprises about 1.25 wt. % to about 10 wt. % of benzoyl peroxide.

In some embodiments, the step of administering comprises topically applying the composition to an area of the skin afflicted with acne lesions. In some embodiments, the area of the skin afflicted with acne lesions is washed prior to application of the composition. In some embodiments, the acne lesions are inflammatory type and/or non-inflammatory type lesions.

In some embodiments, applying the composition results in about a 10%, 20%, 30%, 40%, 50%, 60%, 70%, 80%, 90% or more reduction in number of acne lesions.

In some embodiments, the acne is associated with Propionibacterium acnes.

In some embodiments, the composition is administered to the subject once a day, twice a day, or three times a day.

In some embodiments, the composition is a cream, lotion, gel or emulsion.

In some embodiments, the subject previously exhibited acne lesions.

The present disclosure also provides methods of treating or preventing acne vulgaris, acne necrotica or acne rosacea in a subject in need thereof comprising administering to the subject a composition as described herein.

In some embodiments, the step of administering comprises topically applying the composition to an area of the skin afflicted with acne lesions. In some embodiments, the area of the skin afflicted with acne lesions is first washed prior to application of the composition.

In some embodiments, the acne lesions are inflammatory type and/or non-inflammatory type lesions.

In some embodiments, the composition reduces about 10%, 20%, 30%, 40%, 50%, 60%, 70%, 80%, 90% or more of the acne lesions.

In some embodiments, the acne is associated with Propionibacterium acnes.

In some embodiments, the composition is administered to the subject once a day, twice a day, or three times a day.

These and other embodiments of the invention are described in further detail below.

### BRIEF DESCRIPTION OF THE DRAWINGS

FIG. 1 shows the mean percent change from baseline of an Investigator's Global Assessment (IGA) score by visit and in accordance with an embodiment of the present technology.

FIG. 2 shows the mean percent change from baseline of inflammatory lesion count by visit and in accordance with an embodiment of the present technology.

FIG. 3 shows the mean percent change from baseline of non-inflammatory lesion count by visit and in accordance with an embodiment of the present technology.

FIG. 4 shows the mean percent change from baseline of inflammatory and non-inflammatory lesion count by visit and in accordance with an embodiment of the present technology.

### DETAILED DESCRIPTION

While the present disclosure is capable of being embodied in various forms, the description below of several embodiments is made with the understanding that the present disclosure is to be considered as an exemplification of the disclosure, and is not intended to limit the disclosure to the specific embodiments illustrated. Headings are provided for convenience only and are not to be construed to limit the disclosure in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present disclosure.

In some embodiments, the present disclosure provides compositions comprising one or more of ascorbyl palmitate, tocopherol, ascorbic acid and lecithn. In some embodiments, ascorbyl palmitate, tocopherol, ascorbic acid and lecithin are each, if present, present in an amount of about 0.01% to about 5%, about 0.05% to about 2.5% or about 0.075% to about 1%, by weight of the composition.

The present disclosure also provides methods for treating or preventing acne in a subject in need thereof comprising administering to the subject a composition as described herein.

In one embodiment, compositions provided herein comprise about 0.1 wt. % to about 5 wt. % of ascorbyl palmitate, for example about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. %.

In one embodiment, compositions provided herein comprise about 0.1 wt. % to about 5 wt. % of tocopherol, for example about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. %.

In one embodiment, compositions provided herein comprise about 0.1 wt. % to about 5 wt. % of lecithin (e.g. soy lecithin), for example about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. %.

In one embodiment, compositions provided herein comprise about 0.1 wt. % to about 5 wt. % of ascorbic acid, for example about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. %.

In some embodiments, compositions of the invention may also comprise one or more of steareth-2, steareth-21, cetyl alcohol, medium chain triglycerides, myristyl myristate, isopropyl myristate, methyl 4-hydroxybenzoate, propyl 4 hydroxybenzoate, bronopol, carbomer, xanthan gum, potassium hydroxide, fragrance and water.

In some embodiments, one or more of steareth-2, steareth-21, cetyl alcohol, medium chain triglycerides, myristyl myristate, isopropyl myristate, methyl 4-hydroxybenzoate, propyl 4 hydroxybenzoate, bronopol, carbomer, xanthan gum, and/or potassium hydroxide are present in individual amounts of about 0.01 wt. % to about 5 wt. %, for example about 0.01 wt. %, 0.02 wt. %, 0.03 wt. %, 0.04 wt. %, 0.05 wt. %, 0.06 wt. %, 0.07 wt. %, 0.08, 0.09 wt. %, 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. %. In some embodiments, water is present in an amount of about 30% to about 95%, about 40% to about 90%, about 50% to about 85%, for example about 75%, about 80% or about 85%.

In one embodiment, the composition further comprises an additional active agent. In one embodiment, the composition comprises an amount of the additional active agent that is less than the generally recognized therapeutically effective amount for that agent. In one embodiment, the composition comprises an amount of the additional active agent that is equal to or greater than the generally recognized therapeutically effective amount for that agent. In one embodiment, the additional active agent has not previously been recognized as effective in the treatment or prevention of acne. In another embodiment, the additional active agent is approved for use in the treatment or prevention of acne. In one embodiment, the additional active agent is a peroxide.

In one embodiment, the additional active agent is benzoyl peroxide. In one embodiment, the composition comprises an amount of benzoyl peroxide that is less than the generally recognized therapeutically effective amount. In one embodiment, the composition comprises an amount of benzoyl peroxide that is equal to or greater than the generally recognized therapeutically effective amount. In one embodiment, the composition comprises about 2.5 wt. % to about 10 wt. % of benzoyl peroxide, for example about 1.25 wt. %, about 1.3 wt. %, about 1.4 wt. %, about 1.5 wt. %, about 1.6 wt %, about 1.7 wt %, about 1.7 wt %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, about 8 wt. %, about 8.1 wt. %, about 8.2 wt. %, about 8.3 wt. %, about 8.4 wt. %, about 8.5 wt. %, about 8.6 wt. %, about 8.7 wt. %, about 8.8 wt. %, about 8.9 wt. %, about 9 wt. %, about 9.1 wt. %, about 9.2 wt. %, about 9.3 wt. %, about 9.4 wt. %, about 9.5 wt. %, about 9.6 wt. %, about 9.7 wt. %, about 9.8 wt. %, about 9.9 wt. %, or about 10 wt. % of benzoyl peroxide.

In one embodiment, the additional active agent is adapalene (6-[3-(1-adamantyl)-4-methoxyphenyl]-2-naphthoic acid). In one embodiment, the composition comprises an amount of adapalene that is less than the generally recognized therapeutically effective amount. In one embodiment, the composition comprises an amount of the adapalene that is equal to or greater than the generally recognized therapeutically effective amount. In one embodiment, the composition comprises about 0.05 wt. % to about 0.3 wt. % of adapalene, for example about 0.05 wt. %, about 0.06 wt. %, about 0.07 wt %, about 0.08% about 0.09 wt. %, about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.21 wt. %, about 0.22 wt. %, about 0.23 wt. %, about 0.24 wt. %, about 0.25 wt. %, about 0.26 wt. %, about 0.27 wt. %, about 0.28 wt. %, about 0.29 wt. %, or about 0.3 wt. % of adapalene.

Any pharmaceutically acceptable excipient known to those of skill in the art may be used in compositions according to the present disclosure. Any excipient selected for use in the therapeutic and cosmetic compositions should be pharmaceutically and/or cosmetically acceptable and appropriate for the form in which the therapeutic composition will be used, e.g., cream, gel, milk, oil, lotion, and the like. Preferably, the excipient has an affinity for the skin, is well tolerated, and stable when used in an amount adequate to provide the desired consistency and ease of application. By way of example only, a composition according to the present disclosure may comprise one or more of: surfactants, preservatives, flavoring agents, co-solvents, viscosity aids, suspension aids, and lipophilic phases.

In one embodiment, the composition comprises a stabilizer such as a cetyl alcohol or a saturated cetyl alcohol (e.g., cetyl alcohol). In one embodiment, the composition comprises about 0.1 wt. % to about 5 wt. % of a stabilizer, for example about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.21 wt. %, about 0.22 wt. %, about 0.23 wt. %, about 0.24 wt. %, about 0.25 wt. %, about 0.26 wt. %, about 0.27 wt. %, about 0.28 wt. %, about 0.29 wt. %, about 0.3 wt. %, about 0.31 wt. %, about 0.32 wt. %, about 0.33 wt. %, about 0.34 wt. %, about 0.35 wt. %, about 0.36 wt. %, about 0.37 wt. %, about 0.38 wt. %, about 0.39 wt. %, about 0.4 wt. %, about 0.41 wt. %, about 0.42 wt. %, about 0.43 wt. %, about 0.44 wt. %, about 0.45 wt. %, about 0.46 wt. %, about 0.47 wt. %, about 0.48 wt. %, about 0.49 wt. %, about 0.5 wt. %, about 0.51 wt. %, about 0.52 wt. %, about 0.53 wt. %, about 0.54 wt. %, about 0.55 wt. %, about 0.56 wt. %, about 0.57 wt. %, about 0.58 wt. %, about 0.59 wt. %, about 0.6 wt. %, about 0.61 wt. %, about 0.62 wt. %, about 0.63 wt. %, about 0.64 wt. %, about 0.65 wt. %, about 0.66 wt. %, about 0.67 wt. %, about 0.68 wt. %, about 0.69 wt. %, about 0.7 wt. %, about 0.71 wt. %, about 0.72 wt. %, about 0.73 wt. %, about 0.74 wt. %, about 0.75 wt. %, about 0.76 wt. %, about 0.77 wt. %, about 0.78 wt. %, about 0.79 wt. %, about 0.8 wt. %, about 0.81 wt. %, about 0.82 wt. %, about 0.83 wt. %, about 0.84 wt. %, about 0.85 wt. %, about 0.86 wt. %, about 0.87 wt. %, about 0.88 wt. %, about 0.89 wt. %, about 0.9 wt. %, about 0.91 wt. %, about 0.92 wt. %, about 0.93 wt. %, about 0.94 wt. %, about 0.95 wt. %, about 0.96 wt. %, about 0.97 wt. %, about 0.98 wt. %, about 0.99 wt. %, about 1 wt. %, about 1.01 wt. %, about 1.02 wt. %, about 1.03 wt. %, about 1.04 wt. %, about 1.05 wt. %, about 1.06 wt. %, about 1.07 wt. %, about 1.08 wt. %, about 1.09 wt. %, about 1.1 wt. %, about 1.11 wt. %, about 1.12 wt. %, about 1.13 wt. %, about 1.14 wt. %, about 1.15 wt. %, about 1.16 wt. %, about 1.17 wt. %, about 1.18 wt. %, about 1.19 wt. %, about 1.2 wt. %, about 1.21 wt. %, about 1.22 wt. %, about 1.23 wt. %, about 1.24 wt. %, about 1.25 wt. %, about 1.26 wt. %, about 1.27 wt. %, about 1.28 wt. %, about 1.29 wt. %, about 1.3 wt. %, about 1.31 wt. %, about 1.32 wt. %, about 1.33 wt. %, about 1.34 wt. %, about 1.35 wt. %, about 1.36 wt. %, about 1.37 wt. %, about 1.38 wt. %, about 1.39 wt. %, about 1.4 wt. %, about 1.41 wt. %, about 1.42 wt. %, about 1.43 wt. %, about 1.44 wt. %, about 1.45 wt. %, about 1.46 wt. %, about 1.47 wt. %, about 1.48 wt. %, about 1.49 wt. %, about 1.5 wt. %, about 1.51 wt. %, about 1.52 wt. %, about 1.53 wt. %, about 1.54 wt. %, about 1.55 wt. %, about 1.56 wt. %, about 1.57 wt. %, about 1.58 wt. %, about 1.59 wt. %, about 1.6 wt. %, about 1.61 wt. %, about 1.62 wt. %, about 1.63 wt. %, about 1.64 wt. %, about 1.65 wt. %, about 1.66 wt. %, about 1.67 wt. %, about 1.68 wt. %, about 1.69 wt. %, about 1.7 wt. %, about 1.71 wt. %, about 1.72 wt. %, about 1.73 wt. %, about 1.74 wt. %, about 1.75 wt. %, about 1.76 wt. %, about 1.77 wt. %, about 1.78 wt. %, about 1.79 wt. %, about 1.8 wt. %, about 1.81 wt. %, about 1.82 wt. %, about 1.83 wt. %, about 1.84 wt. %, about 1.85 wt. %, about 1.86 wt. %, about 1.87 wt. %, about 1.88 wt. %, about 1.89 wt. %, about 1.9 wt. %, about 1.91 wt. %, about 1.92 wt. %, about 1.93 wt. %, about 1.94 wt. %, about 1.95 wt. %, about 1.96 wt. %, about 1.97 wt. %, about 1.98 wt. %, about 1.99 wt. %, about 2 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt % of the stabilizer. In one embodiment, the stabilizer is cetyl alcohol (e.g., Crodacol C95 EP, Croda International plc).

In one embodiment, the composition comprises one or more antioxidants such as ascorbic acid, palmitic acid, ascorbyl palmitate, a-tocopherol, idebenone, ubiquinone, ferulic acid, coenzyme Q10, lycopene, green tea, catechins, epigallocatechin 3-gallate (EGCG), green tea polyphenols (GTP), silymarin, coffeeberry, resveratrol, grape seed, pomegranate extracts, genisten, pycnogenol, niacinamide, and the like. In one embodiment, the composition comprises about 0.01 wt. % to about 2 wt. % of an antioxidant, for example about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.21 wt. %, about 0.22 wt. %, about 0.23 wt. %, about 0.24 wt. %, about 0.25 wt. %, about 0.26 wt. %, about 0.27 wt. %, about 0.28 wt. %, about 0.29 wt. %, about 0.3 wt. %, about 0.31 wt. %, about 0.32 wt. %, about 0.33 wt. %, about 0.34 wt. %, about 0.35 wt. %, about 0.36 wt. %, about 0.37 wt. %, about 0.38 wt. %, about 0.39 wt. %, about 0.4 wt. %, about 0.41 wt. %, about 0.42 wt. %, about 0.43 wt. %, about 0.44 wt. %, about 0.45 wt. %, about 0.46 wt. %, about 0.47 wt. %, about 0.48 wt. %, about 0.49 wt. %, about 0.5 wt. %, about 0.51 wt. %, about 0.52 wt. %, about 0.53 wt. %, about 0.54 wt. %, about 0.55 wt. %, about 0.56 wt. %, about 0.57 wt. %, about 0.58 wt. %, about 0.59 wt. %, about 0.6 wt. %, about 0.61 wt. %, about 0.62 wt. %, about 0.63 wt. %, about 0.64 wt. %, about 0.65 wt. %, about 0.66 wt. %, about 0.67 wt. %, about 0.68 wt. %, about 0.69 wt. %, about 0.7 wt. %, about 0.71 wt. %, about 0.72 wt. %, about 0.73 wt. %, about 0.74 wt. %, about 0.75 wt. %, about 0.76 wt. %, about 0.77 wt. %, about 0.78 wt. %, about 0.79 wt. %, about 0.8 wt. %, about 0.81 wt. %, about 0.82 wt. %, about 0.83 wt. %, about 0.84 wt. %, about 0.85 wt. %, about 0.86 wt. %, about 0.87 wt. %, about 0.88 wt. %, about 0.89 wt. %, about 0.9 wt. %, about 0.91 wt. %, about 0.92 wt. %, about 0.93 wt. %, about 0.94 wt. %, about 0.95 wt. %, about 0.96 wt. %, about 0.97 wt. %, about 0.98 wt. %, about 0.99 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, or about 2 wt. % of the one or more antioxidant.

In one embodiment the antioxidant is ascorbyl palmitate. In one embodiment the antioxidant is a-tocopherol. In one embodiment the antioxidant is ascorbic acid. In one embodiment the antioxidant is idebenone. In one embodiment, the antioxidant is ubiquinone. In one embodiment, the antioxidant is ferulic acid. In one embodiment, the antioxidant is coenzyme Q10. In one embodiment, the antioxidant is lycopene. In one embodiment, the antioxidant is green tea. In one embodiment, the antioxidant is catechins. In one embodiment, the antioxidant is epigallocatechin 3-gallate (EGCG). In one embodiment, the antioxidant is green tea polyphenols (GTP). In one embodiment, the antioxidant is silymarin. In one embodiment, the antioxidant is coffeeberry. In one embodiment, the antioxidant is resveratrol. In one embodiment, the antioxidant is grape seed. In one embodiment, the antioxidant is pomegranate extracts. In one embodiment, the antioxidant is genisten. In one embodiment, the antioxidant is pycnogenol. In one embodiment, the antioxidant is niacinamide. In one embodiment, the composition comprises about 0.01 wt. % to about 0.5 wt. % of one or more antioxidants selected from the group consisting of ascorbic acid, palmitic acid, ascorbyl palmitate, a-tocopherol, idebenone, ubiquinone, ferulic acid, coenzyme Q10, lycopene, green tea, catechins, epigallocatechin 3-gallate (EGCG), green tea polyphenols (GTP), silymarin, coffeeberry, resveratrol, grape seed, pomegranate extracts, genisten, pycnogenol, and niacinamide. In one embodiment, the composition comprises about 0.1 wt. % to about 0.3 wt. % of one or more antioxidants selected from the group consisting of ascorbic acid, palmitic acid, ascorbyl palmitate, a-tocopherol, idebenone, ubiquinone, ferulic acid, coenzyme Q10, lycopene, green tea, catechins, epigallocatechin 3-gallate (EGCG), green tea polyphenols (GTP), silymarin, coffeeberry, resveratrol, grape seed, pomegranate extracts, genisten, pycnogenol, and niacinamide. In one embodiment, the composition comprises about 0.3 wt. % to about 0.5 wt. % of one or more antioxidants selected from the group consisting of ascorbic acid, palmitic acid, ascorbyl palmitate, a-tocopherol, idebenone, ubiquinone, ferulic acid, coenzyme Q10, lycopene, green tea, catechins, epigallocatechin 3-gallate (EGCG), green tea polyphenols (GTP), silymarin, coffeeberry, resveratrol, grape seed, pomegranate extracts, genisten, pycnogenol, and niacinamide. In one embodiment, the composition comprises about 0.45 wt. % of one or more antioxidants selected from the group consisting of ascorbic acid, palmitic acid, ascorbyl palmitate, a-tocopherol, idebenone, ubiquinone, ferulic acid, coenzyme Q10, lycopene, green tea, catechins, epigallocatechin 3-gallate (EGCG), green tea polyphenols (GTP), silymarin, COFFEEBERRY, resveratrol, grape seed, pomegranate extracts, genisten, pycnogenol, and niacinamide. In one embodiment, the composition comprises about 0.05 wt. % of idebenone. In one embodiment, the composition comprises about 0.05 wt. % to about 1 wt. % of ubiquinone, for example about 0.05 wt. %, about 0.1 wt. %, about 0.15 wt. %, about 0.2 wt. %, about 0.25 wt. %, about 0.3 wt. %, about 0.35 wt. %, about 0.4 wt. %, about 0.45 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, or about 1 wt. % of ubiquinone. In one embodiment, the composition comprises about 0.1 wt. % to about 1 wt. % of ferulic acid, for example about 0.1 wt. %, about 0.15 wt. %, about 0.2 wt. %, about 0.25 wt. %, about 0.3 wt. %, about 0.35 wt. %, about 0.4 wt. %, about 0.45 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, or about 1 wt. % of ferulic acid. In one embodiment, the composition comprises about 0.01 wt. % to about 0.5 wt. % of ascorbyl palmitate, about 0.01 wt. % to about 0.5 wt. % of a-tocopherol, and about 0.01 wt. % to about 0.5 wt. % of ascorbic acid. In one embodiment the composition comprises about 0.1 wt. % to about 0.3 wt. % of ascorbyl palmitate, about 0.1 wt. % to about 0.3 wt. % of a-tocopherol, and about 0.05 wt. % to about 0.2 wt. % of ascorbic acid. In one embodiment the composition comprises about 0.2 wt. % of ascorbyl palmitate, about 0.15 wt. % of a-tocopherol, and about 0.1 wt. % of ascorbic acid.

In one embodiment, the composition comprises one or more emollients such as a fully saturated triglyceride (e.g., medium-chain triglycerides such as Crodamol GTCC, Croda International plc), myristyl myristate, isopropryl palmitate, and glycerin. In one embodiment, the composition comprises about 0.5 wt. % to about 20 wt. % of an emollient, for example about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, about 8 wt. %, about 8.1 wt. %, about 8.2 wt. %, about 8.3 wt. %, about 8.4 wt. %, about 8.5 wt. %, about 8.6 wt. %, about 8.7 wt. %, about 8.8 wt. %, about 8.9 wt. %, about 9 wt. %, about 9.1 wt. %, about 9.2 wt. %, about 9.3 wt. %, about 9.4 wt. %, about 9.5 wt. %, about 9.6 wt. %, about 9.7 wt. %, about 9.8 wt. %, about 9.9 wt. %, about 10 wt. %, about 10.1 wt. %, about 10.2 wt. %, about 10.3 wt. %, about 10.4 wt. %, about 10.5 wt. %, about 10.6 wt. %, about 10.7 wt. %, about 10.8 wt. %, about 10.9 wt. %, about 11 wt. %, about 11.1 wt. %, about 11.2 wt. %, about 11.3 wt. %, about 11.4 wt. %, about 11.5 wt. %, about 11.6 wt. %, about 11.7 wt. %, about 11.8 wt. %, about 11.9 wt. %, about 12 wt. %, about 12.1 wt. %, about 12.2 wt. %, about 12.3 wt. %, about 12.4 wt. %, about 12.5 wt. %, about 12.6 wt. %, about 12.7 wt. %, about 12.8 wt. %, about 12.9 wt. %, about 13 wt. %, about 13.1 wt. %, about 13.2 wt. %, about 13.3 wt. %, about 13.4 wt. %, about 13.5 wt. %, about 13.6 wt. %, about 13.7 wt. %, about 13.8 wt. %, about 13.9 wt. %, about 14 wt. %, about 14.1 wt. %, about 14.2 wt. %, about 14.3 wt. %, about 14.4 wt. %, about 14.5 wt. %, about 14.6 wt. %, about 14.7 wt. %, about 14.8 wt. %, about 14.9 wt. %, about 15 wt. %, about 15.1 wt. %, about 15.2 wt. %, about 15.3 wt. %, about 15.4 wt. %, about 15.5 wt. %, about 15.6 wt. %, about 15.7 wt. %, about 15.8 wt. %, about 15.9 wt. %, about 16 wt. %, about 16.1 wt. %, about 16.2 wt. %, about 16.3 wt. %, about 16.4 wt. %, about 16.5 wt. %, about 16.6 wt. %, about 16.7 wt. %, about 16.8 wt. %, about 16.9 wt. %, about 17 wt. %, about 17.1 wt. %, about 17.2 wt. %, about 17.3 wt. %, about 17.4 wt. %, about 17.5 wt. %, about 17.6 wt. %, about 17.7 wt. %, about 17.8 wt. %, about 17.9 wt. %, about 18 wt. %, about 18.1 wt. %, about 18.2 wt. %, about 18.3 wt. %, about 18.4 wt. %, about 18.5 wt. %, about 18.6 wt. %, about 18.7 wt. %, about 18.8 wt. %, about 18.9 wt. %, about 19 wt. %, about 19.1 wt. %, about 19.2 wt. %, about 19.3 wt. %, about 19.4 wt. %, about 19.5 wt. %, about 19.6 wt. %, about 19.7 wt. %, about 19.8 wt. %, about 19.9 wt. %, or about 20 wt. % of an emollient. In one embodiment, the composition comprises about 0.5 wt. % to about 5 wt. % of any one emollient. In one embodiment, the one or more emollients are selected from the group consisting of medium-chain triglycerides (e.g., Crodamol GTCC, Croda International plc), myristyl myristate, isopropryl palmitate, and glycerin.

In one embodiment, the composition comprises medium-chain triglycerides (e.g., Crodamol GTCC), myristyl myristate, isopropryl palmitate and glycerin in a combined amount of about 0.5 wt. to about 20 wt. %. In one embodiment, the composition comprises about 0.5 wt. % to about 5 wt. % of medium-chain triglycerides (e.g., Crodamol GTCC), for example about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of medium-chain triglycerides (e.g., Crodamol GTCC). In one embodiment, the composition comprises about 0.5 wt. % to about 5 wt. % of myristyl myristate, for example about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of myristyl myristate.

In one embodiment, the composition comprises about 0.5 wt. % to about 8 wt. % of isopropryl palmitate, for example about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, or about 8 wt. % of isopropryl palmitate.

In one embodiment, the composition comprises about 0.5 wt. % to about 5 wt. % of glycerin, for example about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of glycerin. in one embodiment, the composition comprises about 2 wt. % of medium-chain triglycerides (e.g., Crodamol GTCC), about 2 wt. % of myristyl myristate (e.g., Crodamol MM, Croda International plc), about 4 wt. % of isopropryl palmitate (e.g., Crodamol IPP, Croda International plc), and about 1 wt. % of glycerin.

In one embodiment, the composition comprises a preservative such as phenoxyethanol. In one embodiment, the composition comprises about 0.1 wt. % to about 5 wt. % of a preservative, for example about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of a preservative. In one embodiment, the preservative is phenoxyethanol. In one embodiment, the composition comprises about 0.5 wt. % to about 5 wt. % of phenoxyethanol. In one embodiment, the composition comprises about 0.5 wt. % to about 2 wt. % of phenoxyethanol. In one embodiment, the composition comprises about 1 wt. % of phenoxyethanol.

In one embodiment, the composition comprises one or more thickeners, such as a cross-linked polymer (e.g., a cross-linked acrylic acid polymer such as carbomer, available commercially as Carbopol ETD2020NF, Lubrizol Corp.), a polysaccharide (e.g., a xanthan gum such as CPKelko's Keltrol 11K). In one embodiment, the composition comprises about 0.1 wt. % to about 5 wt. % of one or more thickeners, for example about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of one or more thickeners. In one embodiment, the one or more thickeners is one or more of a cross-linked acrylic acid polymer and a polysaccharide. In one embodiment, the one or more thickeners are Carbopol ETD2020NF and Keltrol 11K. In one embodiment, the composition comprises about 0.1 wt. % to about 5 wt. % of Carbopol ETD2020NF and about 0.1 wt. % to about 5 wt. % of Keltrol 11K. In one embodiment, the composition comprises about 0.5 wt. % to about 1 wt. % of Carbopol ETD2020NF and about 0.2 wt. % to about 1 wt. % of Keltrol 11K. In one embodiment, the composition comprises about 0.8 wt. % of Carbopol ETD2020NF and about 0.4 wt. % of Keltrol 11K.

In one embodiment, the composition comprises one or more texturizers such as a lecithin (e.g., a liquid soy lecithin such as Leciprime 1400 IPM, Cargill, Inc.). In one embodiment, the composition comprises about 0.1 wt. % to about 5 wt. % of one or more texturizers, for example about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of one or more texturizers. In one embodiment, the one or more texturizers comprise Leciprime 1400 IPM. In one embodiment, the composition comprises about 0.1 wt. % to about 5 wt. % of Leciprime 1400 IPM. In one embodiment, the composition comprises about 0.2 wt. % to about 1 wt. % of Leciprime 1400 IPM. In one embodiment, the composition comprises about 0.5 wt. % of Leciprime 1400 IPM.

In one embodiment, the composition comprises one or more fragrances such as Floral Spa 760, Sensual Wood 138 or Mild Care 345. In one embodiment, the composition comprises about 0.01 wt. % to about 0.5 wt. % of one or more fragrances, for example about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.21 wt. %, about 0.22 wt. %, about 0.23 wt. %, about 0.24 wt. %, about 0.25 wt. %, about 0.26 wt. %, about 0.27 wt. %, about 0.28 wt. %, about 0.29 wt. %, about 0.3 wt. %, about 0.31 wt. %, about 0.32 wt. %, about 0.33 wt. %, about 0.34 wt. %, about 0.35 wt. %, about 0.36 wt. %, about 0.37 wt. %, about 0.38 wt. %, about 0.39 wt. %, about 0.4 wt. %, about 0.41 wt. %, about 0.42 wt. %, about 0.43 wt. %, about 0.44 wt. %, about 0.45 wt. %, about 0.46 wt. %, about 0.47 wt. %, about 0.48 wt. %, about 0.49 wt. %, or about 0.5 wt. % of one or more fragrances. In one embodiment, the composition comprises about 0.01 wt. % to about 0.5 wt. % of Mild Care 345 fragrance. In one embodiment, the composition comprises about 0.01 wt. % to about 0.1 wt. % of Mild Care 345 fragrance. In one embodiment, the composition comprises about 0.01 wt. % to about 0.05 wt. % of Mild Care 345 fragrance. In one embodiment, the composition comprises about 0.05 wt. % of Mild Care 345 fragrance.

A composition for use in accordance with the disclosure can be formulated as one or more dosage units. The terms “dose unit” and “dosage unit” herein refer to a portion of a composition that contains an amount of a therapeutic agent suitable for a single administration to provide a therapeutic effect. Such dosage units may be administered one to a plurality (i.e. 1 to about 10, 1 to 8, 1 to 6, 1 to 4 or 1 to 2) of times per day, or as many times as needed to elicit a therapeutic response.

In one embodiment, a composition including, for example, a composition, as disclosed herein is formulated as an aerosol, a gel, an ointment, a lotion, a cream, a gel stick, a liniment, or a spray.

The compositions and formulations disclosed herein may be used in the treatment of diseases and/or disorders including, for example, disease and/or disorders of the skin such as acne, atopic dermatitis, pluritus, etc.

Methods are provided herein for treating or preventing acne (e.g., acne vulgaris and/or acne necrotica) in a subject in need thereof comprising administering to the subject a composition as described herein.

The term “acne” herein refers to any disease or disorder of the skin that presents with one or more acneiform eruptions such as papules, pustules, cysts, and the like. Non-limiting examples of acne include acne vulgaris, acne necrotica, halogen acne, chloracne, occupational acne, oil acne, tar acne, acne aestivalis, tropical acne, acne cosmetica, pomade acne, acne keloidalis nuchae, acne mechanica, excoriated acne, acne medicamentosa, infantile acne, neonatal acne, acne conglobata, acne fulminans, acne miliaris necrotica, miliaris disseminatus faciei, and, and other skin disorders associated with acneiform eruptions.

In one embodiment, the present disclosure provides a method of treating or preventing acne associated with P. acnes in a subject in need thereof. In one embodiment, the method comprises administering to the subject a composition as disclosed herein.

In one embodiment, the present disclosure provides a method of inhibiting P. acnes including, for example, its growth, colonization and/or infection in a subject in need thereof. In one embodiment, the method comprises contacting P. acnes with a composition as disclosed herein.

In one embodiment, the method further comprises washing an affected area of the skin (and/or to an area of the skin that is generally prone to development of acneiform eruptions) prior to administering the composition. As used herein, the term “washing” refers generally to any method known to those of skill in the art for cleansing the skin, exfoliating the skin, removing dirt, oil, dead skin cells and the like from the skin, etc.

In one embodiment, the method comprises topically administering the composition to an area of the skin affected with acne lesions and/or to an area of the skin that is generally prone to development of acne lesions and/or previously had acne lesions.

In one embodiment, the method comprises topically administering the composition to an area of the skin affected with non-inflammatory acne lesions. In one embodiment, the method comprises topically administering the composition to an area of the skin affected with inflammatory acne lesions. In one embodiment, the method comprises topically administering the composition to an area of the skin affected with both non-inflammatory and inflammatory acne lesions.

In one embodiment, the method comprises administering a composition as disclosed herein once per day, twice per day, three times per day, or more than three times per day.

In one embodiment, upon treatment in accordance with the present disclosure, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the treated area of the skin comprises about 10%, about 20%, about 30%, about 40%, about 50%, about 60%, about 70%, about 80%, about 90%, or greater than about 90% fewer acne lesions than before treatment.

As used herein, “treating” or “treatment” of a disease, disorder, or condition includes at least partially: (1) preventing the disease, disorder, or condition, i.e. causing the clinical symptoms of the disease, disorder, or condition not to develop in a mammal that is exposed to or predisposed to the disease, disorder, or condition but does not yet experience or display symptoms of the disease, disorder, or condition; (2) inhibiting the disease, disorder, or condition, i.e., arresting or reducing the development of the disease, disorder, or condition or its clinical symptoms; or (3) relieving the disease, disorder, or condition, i.e., causing regression of the disease, disorder, or condition or its clinical symptoms. The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

An “effective amount,” as used herein, refers to the amount of an active composition that is required to confer a therapeutic effect on the subject. A “therapeutically effective amount,” as used herein, refers to a sufficient amount of an agent or a compound being administered which will relieve to some extent one or more of the symptoms of the disease, disorder, or condition being treated. In some embodiments, the result is a reduction and/or alleviation of the signs, symptoms, or causes of a disease, or any other desired alteration of a biological system. For example, in some embodiments, an “effective amount” for therapeutic uses is the amount of the composition including a compound as disclosed herein required to provide a clinically significant decrease in disease symptoms without undue adverse side effects. In some embodiments, an appropriate “effective amount” in any individual case is determined using techniques, such as a dose escalation study. The term “therapeutically effective amount” includes, for example, a prophylactically effective amount. In other embodiments, an “effective amount” of a compound disclosed herein, such as a compound of Formula (A) or Formula (I), is an amount effective to achieve a desired pharmacologic effect or therapeutic improvement without undue adverse side effects. In other embodiments, it is understood that “an effect amount” or “a therapeutically effective amount” varies from subject to subject, due to variation in metabolism, age, weight, general condition of the subject, the condition being treated, the severity of the condition being treated, and the judgment of the prescribing physician. The term “pharmaceutically acceptable” in the present context means that the substance in question does not produce unacceptable toxicity to the subject or interaction with other components of the composition.

In another embodiment, the present disclosure provides a method of slowing progression of or promoting regression of acne vulgaris and/or acne necrotica in a subject in need thereof, comprising administering to a subject in need thereof one or more compositions as disclosed herein.

In one embodiment, the present disclosure provides a method of reducing production of sebum in at least a portion of a subject's skin. In one embodiment, the method comprises administering to the subject a therapeutically effective amount of a composition as disclosed herein. In one embodiment, the amount of sebum produced per square inch for a given affected area of the subject's skin after administration of a composition as disclosed herein is less than, or substantially less than the amount of sebum produced before administration of a composition as disclosed herein. In one embodiment, treatment according to the present method results in a 10% reduction, about a 20% reduction, about a 30% reduction, about a 40% reduction, about a 50% reduction, about a 60% reduction, about a 70% reduction, about a 80% reduction, about a 90% reduction, or more than a 90% reduction in sebum production for a given area of the subject's skin. In one embodiment, the reduction in sebum production occurs within about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months of the initiation of the treatment method.

In one embodiment, the present disclosure provides a method of reducing acne scarring in at least a portion of a subject's skin. In one embodiment, the method comprises administering to the subject a therapeutically effective amount of a composition as disclosed herein. In one embodiment, the amount of acne-related scarring per square inch for a given affected area of the subject's skin after administration of a composition as disclosed herein is less than, or substantially less than the amount of acne-related scarring present in the same area of skin before administration of a composition as disclosed herein. In one embodiment, treatment according to the present method results in a 10% reduction, about a 20% reduction, about a 30% reduction, about a 40% reduction, about a 50% reduction, about a 60% reduction, about a 70% reduction, about a 80% reduction, about a 90% reduction, or more than a 90% reduction in acne-related scarring for a given area of the subject's skin. In one embodiment, the reduction in acne-related scarring occurs within about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months of the initiation of the treatment method.

The present disclosure also provides methods for reducing including, for example preventing, transepidermal water loss (TEWL). TEWL and diseases or disorders associated with TEWL may be identified by determining a TEWL measurement for at least one portion of the skin (see, e.g., Mundlein et al. (2008) Sensors and Actuators A: Physical 142(1):67-72) and comparing the measurement with that of normal skin (e.g., undiseased). The methods for reducing TEWL may comprise administering to a subject in need thereof a therapeutically effective amount of a composition as disclosed herein. Such methods may be useful in the treatment and/or prevention of diseases or disorders associated with a disturbance in the stratum corneum (e.g., atopic dermatitis).

Without further description, it is believed that one of ordinary skill in the art may, using the preceding description and the following illustrative examples, make and utilize the agents of the present disclosure and practice the claimed methods. The following working examples are provided to facilitate the practice of the present disclosure, and are not to be construed as limiting in any way the remainder of the disclosure.

### EXAMPLES

### Example 1

A randomized, placebo-controlled, double-blind, parallel group, multi-center Phase II study was conducted to investigate the efficacy and safety of topically applied compositions and the dose-response relationship among two doses of fatty acid compositions (Test Articles 1 and 2) and a third composition (Test Article 3) in patients with mild to moderate acne vulgaris.

The study consisted of a wash-out period of maximum 14 days; a 12 week treatment period and a 4 week follow up period. At the Screening visit, after giving informed consent to participate, patients were assessed using the screening examinations. Eligible patients with confirmed acne vulgaris and who meet the inclusion criteria and did not meet the exclusion criteria were enrolled.

During the 14 day wash-out period, the treatment period and follow-up period patients were restricted from using any other treatment for acne vulgaris, or from taking or any antimicrobial medication for the treatment of acne. Any medication (prescription as well as over the counter (OTC) drugs) or therapeutic intervention deemed necessary for the patient, and which in the opinion of the Investigator would not interfere with the safety and efficacy evaluations, were allowed to be continued unless they were included in the list of ‘medications and therapeutic regimens excluded from the study.

Following completion of the wash-out period, before the comparative treatment period commenced, patients returned to the site for a final investigator assessment of the severity of their acne vulgaris and eligible patients were randomly allocated to one of the three parallel group treatment regimens to a 1:1:1 randomization:

Test Article 1 applied topically to all affected areas twice-daily for 12 weeks;

Test Article 2 applied topically to all affected areas twice-daily for 12 weeks; or

Test Article 3 applied topically to all affected areas twice-daily for 12 weeks.

The Composition of Test Article 3 is shown below:

| Chemical/Monograph | Formulation |
|---|---|
| Description | (g/100 g) |
| Steareth-2 | 1.65 |
| Steareth-21 | 1.35 |
| Cetyl Alcohol | 0.5 |
| L(+) Ascorbyl Palmitate | 0.2 |
| all-rac-α-Tocopherol | 0.15 |
| Medium Chain Triglycerides | 4.0 |
| Myristyl Myristate | 2.0 |
| Isopropyl Palmitate | 4.0 |
| Glycerol, Anhydrous | 4.0 |
| Methyl 4-Hydroxybenzoate | 0.4 |
| Ethyl 4-Hydroxybenzoate | 0.09 |
| Propyl 4-Hydroxybenzoate | 0.06 |
| Bronopol | 0.02 |
| Ascorbic Acid | 0.1 |
| Carbomer | 0.8 |
| Xanthan Gum | 0.4 |
| Soy Lecithin | 0.5 |
| Potassium Hydroxide | 0.4 |
| Fragrance | 0.05 |
| Water For Irrigation | 79.33 |

To maintain the double-blind conditions, all test compositions had identical appearance. All tubes from the previous visit were weighed at weeks 4, 8 and 12 to assess patient compliance.

During the study, six visits to the clinic were scheduled after the screening visit (Visit 1) according to Table 1 below: one visit at the start of the treatment period/baseline (Day 0/Visit 2), four visits in the treatment period; Week2/Visit 3, Week4/Visit 4, Week 8/Visit 5 and Week 12/Visit 6 and a final safety follow-up visit (Visit 7) 4 weeks after Visit 6 or 28 days after the final visit attended if the patient did not complete the study.

TABLE 1
Study Visit Schedule

| Visit 1 | Visit 2 | Visit 3 | Visit 4 | Visit 5 | Visit 6 | Visit 7 |
|---|---|---|---|---|---|---|
| Screening | Baseline | Week 2 | Week 4 | Week 8 | Week 12 | Follow-up |
| (Week—2) | (Day 0) | (Day 14) | (Day 28) | (Day 56) | (Day 84) | (Week 16) |

Following completion of a successful screening visit, and wash-out period, patients began the comparative treatment period (12 weeks—from day 0 to Day 84). Patients were instructed to apply their randomized test composition to the affected area as indicated in the patient information sheet. At the start of the comparative treatment period, after confirmation of continued eligibility, patients were randomly assigned to one of the three treatment regimens.

The Test Articles were topically applied to all affected areas twice daily (morning and evening) by the patients. After washing the face with a mild cleanser, the cream (2-3 fingertip-units per day) were applied twice daily to the clean and dry skin. This amounted to a daily application of 1 gram of cream. The amount of skin to be treated did not exceed 3% of the body surface area. Each self-administration of drug was recorded on a patient diary card. Patients topically applied the composition to all affected areas twice daily for 12 weeks (84 consecutive days).

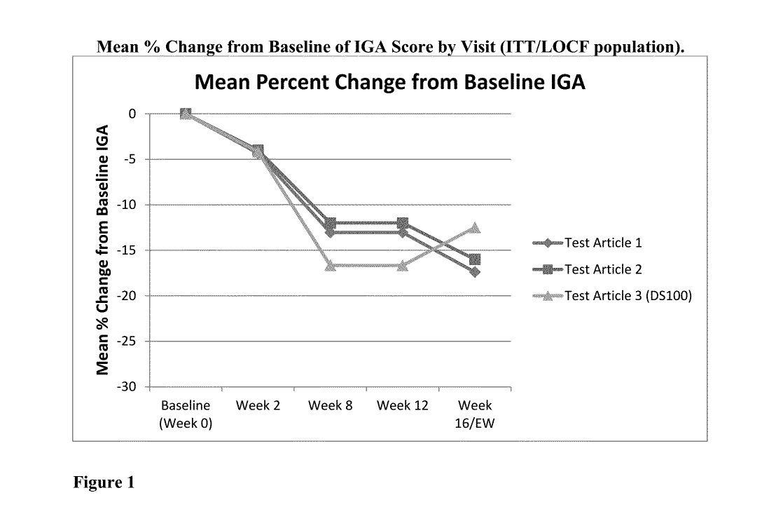

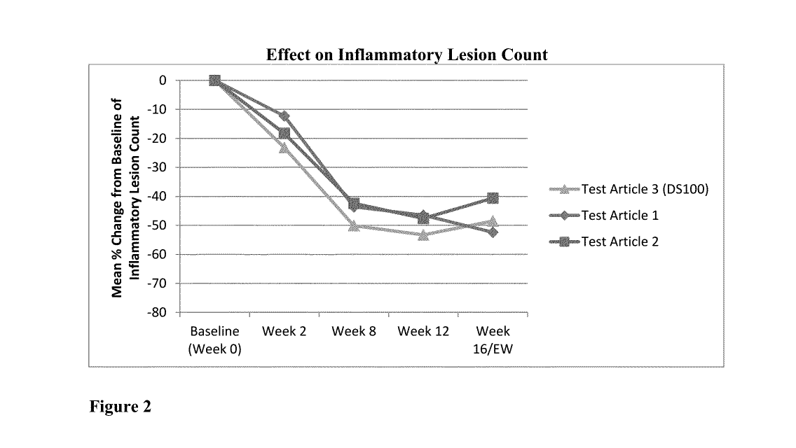

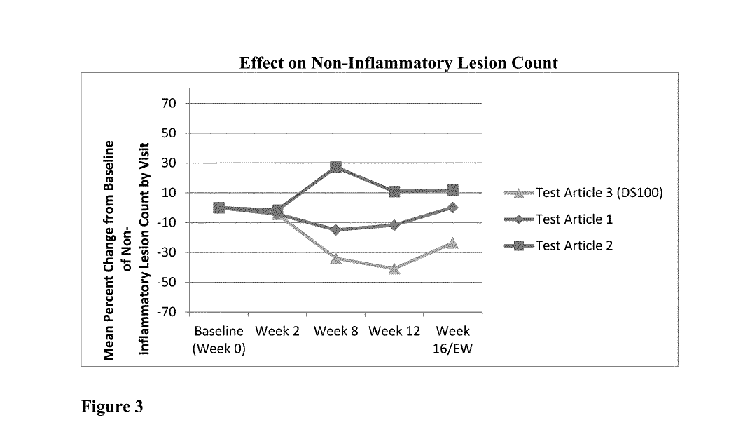

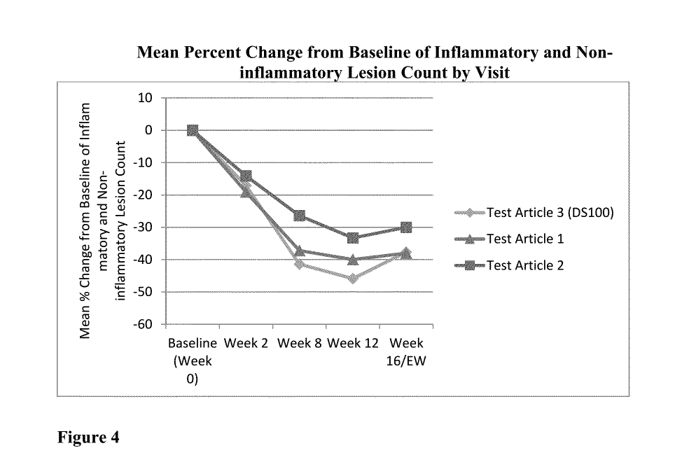

Results are shown in FIGS. 1-4. All of the safety endpoints in the trial were met. Only one SAE was recorded in the trial (a case of acute appendicitis unrelated to study medication). All treatments were very well tolerated locally with <2% experiencing mild local intolerance. Only 3 patients from 154 enrolled were withdrawn from the study due to perceived lack of efficacy.

What is claimed is:

1. A method for treating or preventing a skin condition in a subject in need thereof comprising administering to the subject a composition comprising ascorbyl palmitate, tocopherol, ascorbic acid and lecithin.

2. The method of claim 1 wherein the skin condition is an acne condition.

3. The method of claim 1 wherein the ascorbyl palmitate, tocopherol, ascorbic acid and lecithin are each present in an amount of about 0.01% to about 5%, about 0.05% to about 2.5% or about 0.075% to about 1%, by weight of the composition.

4. A topical composition comprising ascorbyl palmitate, tocopherol, ascorbic acid and lecithin.

5. The composition of claim 4 further comprising one or more excipients.
